(19) United States
(12) Patent Application Publication
(10) Pub. No.: US 2021/0088428 A1
(43) Pub. Date: Mar. 25, 2021

(54) METHOD FOR MEASURING MICRO-SCALE STRENGTH AND RESIDUAL STRENGTH OF BRITTLE ROCK

(71) Applicant: Southwest Petroleum University, Chengdu (CN)

(72) Inventors: Rui SONG, Chengdu (CN); Yao WANG, Chengdu (CN); Mengmeng CUI, Chengdu (CN); Jiajun PENG, Chengdu (CN); Jianjun LIU, Chengdu (CN)

(21) Appl. No.: 16/826,342

(22) Filed: Mar. 23, 2020

(30) Foreign Application Priority Data
Sep. 25, 2019 (CN) 201910911239.7

(51) Int. Cl. G01N 3/08; G01N 23/046; G01N 23/06; G01N 33/24

(52) U.S. Cl. CPC G01N 3/08; G01N 23/046; G01N 23/06; G01N 2223/616; G01N 2203/0019; G01N 2223/04; G01N 2223/419; G01N 33/24

(57) ABSTRACT

A method for measuring micro-scale strength and residual strength of brittle rocks, including: performing micro-CT scanning on a target area; obtaining loading and unloading curves and an elastic modulus of the rock via micro indentation experiment; performing dimensionless analysis based on Buckinham's π-theorem to obtain relation between the loading and unloading curves and elastic modulus, indentation depth, initial and residual strengths; reconstructing a grid model of micro rock matrix at the target area and indenter; performing micro indentation numerical simulation based on Mohr-Coulomb criterion to obtain loading and unloading curves under different strengths and residual strengths; fitting a formula between simulated work of the indenter and initial and residual strengths at h/R of 0.1 and 0.15; and substituting experimental values of the work into the formula to plotting curves of initial and residual strengths under two indentation depths, where coordinates of an intersection point represent micro-scale initial and residual strengths.

### CROSS-REFERENCE TO RELATED APPLICATIONS

This application claims the benefit of priority from Chinese Patent Application No. 201910911239.7, filed on Sep. 25, 2019. The content of the aforementioned application, including any intervening amendments thereto, is incorporated herein by reference in its entirety.

### TECHNICAL FIELD

This application relates to rock mechanics, and more particularly to a method for measuring micro-scale strength and residual strength of brittle rocks.

### BACKGROUND OF THE INVENTION

Rocks are geological carriers for underground construction including tunnels and underground storage, as well as the mineral resources like coal, oil, gas, and geothermal energy. The mechanical properties of rock are essential for the long-term stability of the related constructions and the mining efficiency of the energy resources. As a porous medium cemented by various minerals, the mechanism of micro-scale deformation and cracking and the fluid transport property of rocks have been increasingly investigated in recent years. However, due to the limitations in the determination of a micro rock sample using a conventional rock mechanical device, there is still a lack of a method for effectively measuring micro-scale strength parameters of rocks. Currently, the micro indentation experiments have been performed to evaluate micro mechanical characteristics of the rocks, in which the micro diamond indenters of different shapes are indented into rock minerals to obtain loading and unloading curves of rock minerals. However, due to the complexity and heterogeneity of mineral composition and pore structure of the rock, the current indentation experiment, which mainly focuses on the measurement of elastic modulus and hardness parameters, fails to achieve the determination of strength parameters (such as initial cohesive force and residual cohesive force) of rocks. Therefore, there is an urgent need to develop a method of measuring micro-scale strength and residual strength of brittle rocks to overcome the defects in the prior art.

### SUMMARY OF THE INVENTION

An object of the invention is to provide a method for measuring micro-scale strength and residual strength of brittle rocks to overcome the lack of the determination of micro rock mechanical properties in the art.

In the invention, the failure of brittle rocks is in compliance with the Mohr-Coulomb criterion and the cohesion weakening-friction strengthening principle. Since the micro indentation experiment merely has a micro-scale indentation depth and almost no rock minerals suffer from “shattering”, the internal friction angle of the rock is considered to be constant during this experiment.

The invention provides a method for measuring micro-scale strength and residual strength of brittle rocks, comprising:

(1) performing a dimensionless analysis of a work of the indenter during a loading process of a micro indentation experiment, wherein a shape function Πᵢ, of a loading curve of the micro indentation experiment is expressed as function (1):

Πᵢ=Fᵢ(E,fₚ,α,h,R,fₚₒᵣₑ) (1);

wherein the shape function Πᵢ is affected by an elastic modulus E of a rock sample, a taper angle α of an indenter tip, a radius R of the indenter tip, a plasticity parameter fₚ of a rock material and microstructure characteristics fₚₒᵣₑ of a contact area between the indenter and rock sample;

the work of the indenter is expressed as equation (2):

W=∫₀^{h}^{ₘₐₓ}Fdh (2);

wherein a rock matrix of the brittle rocks is homogeneous and isotropic and meets the Mohr-Coulomb criterion (3):

τₙ=C+σₙ tan φ (3);

wherein τₙ is a shear stress, σₙ is a normal stress, C and φ are an initial cohesive force and an initial internal friction angle of the rock sample, respectively; Cᵣ and φᵣ are a residual cohesive force and a residual internal friction angle of the rock sample after failure, respectively; when the rock sample is not smashed, the micro-scale internal friction angle equals to a core-scale value and C=Cᵣ, so equation (2) is rewritten as equation (4):

W=Fᵢ(E,C,Cᵣ,α,h,R,fₚₒᵣₑ) (4);

wherein the microstructure of the contact area between the indenter and rock sample is reconstructed using micro-CT scanning, and for an indentation experiment using a specially-shaped indenter, the dimensionless analysis of equation (4) is simplified according to the Buckinham's π-theorem as equation (5):

$$\frac{W}{{Ch}^{3}} = {F}_{i} ( \frac{C}{E} , \frac{{C}_{r}}{C} , \frac{h}{R} ) \text{;} \qquad ( 5 )$$

when a feature depth h/R of the indentation experiment is set to 0.1 and 0.15, equation (5) is rewritten as equation (6):

$$Error ! Reference source not found . \frac{W \vert \frac{h}{R} = 0.1 or 0.5}{{Ch}^{3}} = {F}_{i} ( \frac{C}{E} , \frac{{C}_{r}}{C} ) \text{;} \qquad ( 6 )$$

(2) selecting and preparing the rock sample, obtaining the microstructure characteristics via CT scanning and reconstructing a finite element grid model of the rock matrix and the indenter in combination with a digital rock modeling technique;

(3) carrying out the micro indentation experiment on the rock sample, obtaining loading and unloading curves, and calculating micro elastic modulus of the rock according to an indentation experiment specification; and calculating the work of the indenter at h/R respectively of 0.1 and 0.15;

(4) performing a micro indentation numerical simulation for the rock sample under different strengths and residual strengths based on the micro elastic modulus obtained in the micro indentation experiment to obtain loading and unloading curves of the numerical simulation;

(5) calculating a simulated work of the indenter obtained in the numerical simulation at the feature depths h/R respectively of 0.1 and 0.15; and fitting the simulated work of the indenter under different strengths and residual strengths via a cubic polynomial to obtain equation (7):

$$\frac{W}{{Ch}^{3}} = {{A}_{4} [ ln ( \frac{C}{E} ) ]}^{3} + {{A}_{3} [ ln ( \frac{C}{E} ) ]}^{2} + {A}_{2} [ ln ( \frac{C}{E} ) ] + {A}_{1} \text{;} \qquad ( 7 )$$

wherein coefficients A₁˜-A₄ are fitted according to simulation data;

(6) substituting values of the work of the indenter obtained at h/R=0.1 and 0.5 into equation (7) respectively, plotting two curves using the initial cohesive force C as vertical coordinate and the residual cohesive force Cᵣ as horizontal coordinate at the h/R respectively of 0.1 and 0.15, wherein an abscissa and an ordinate of an intersection point of the two curves respectively represent a micro-scale initial cohesive force and a micro-scale residual cohesive force of the rock sample at a detection point; and obtaining the micro-scale strength and residual strength of the rock sample by substituting the C and Cᵣ of the rock sample into equation (3).

As compared with the prior art, the invention has excellent feasibility and high precision.

### BRIEF DESCRIPTION OF THE DRAWINGS

The invention will be further described with reference to the accompanying drawings and embodiments.

FIG. 1 is a flow chart of a method for measuring micro-scale strength and residual strength of rocks according to the invention.

FIG. 2a shows a rock sample S1 according to an embodiment of the invention;

FIG. 2b shows a device for preparing the rock sample S1 according to the embodiment of the invention;

FIG. 2c shows a device used in the micro-CT scanning according to the embodiment of the invention; and

FIG. 2d shows a three-dimensional diagram of the rock sample S1 obtained in the micro-CT scanning according to the embodiment of the invention.

FIG. 3a schematically shows a truncated cone-shaped indenter according to the embodiment of the invention; and

FIG. 3b schematically shows a reconstruction model of a micro rock matrix of the rock sample S1.

FIG. 4 shows loading and unloading curves of the rock sample S1 obtained via a micro indentation experiment according to the embodiment of the invention.

FIG. 5 shows loading curves of the rock sample S1 obtained via a typical micro indentation numerical simulation under different strength characteristics according to the embodiment of the invention.

FIGS. 6a-6b show the relationship between W/Ch³ and Cᵣ/E of the rock sample S1 under different strengths and residual strengths at h/R respectively of 0.1 and 0.15 according to the embodiment of the invention.

FIG. 7 schematically shows the calculation of a micro-scale cohesive force and a micro-scale residual cohesive force of the rock according to the embodiment of the invention.

### DETAILED DESCRIPTION OF EMBODIMENTS

The invention will be described in detail below with reference to the accompanying drawings and embodiments to make the technical solutions, objects and advantages of the invention clearer. It should be understood that described below are merely preferred embodiments of the invention and are not intended to limit the invention. Other embodiments made by those skilled in the art based on the content disclosed herein without paying any creative effort should fall within the scope of the invention.

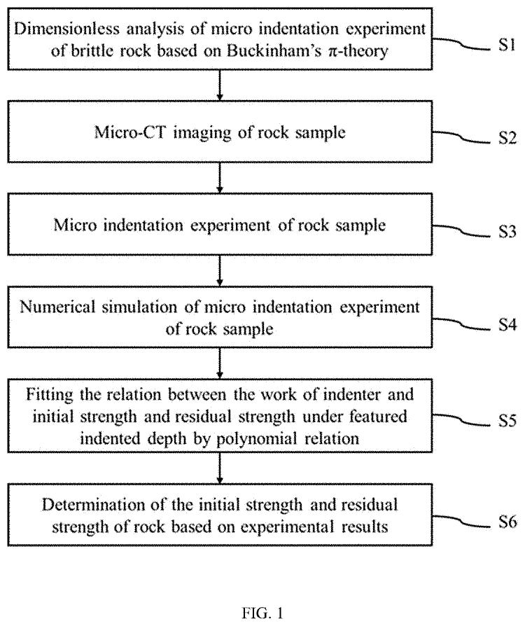

As shown in FIG. 1, the procedure of the proposed method for measuring micro-scale strength and residual strength of brittle rocks includes the following steps.

(1) A dimensionless analysis of a work of the indenter of brittle rocks during a loading process of a micro indentation experiment is performed.

According to a shape function Πᵢ of a loading curve of the micro indentation experiment and based on the Buckinham's π-theorem, a dimensionless function of the work of the indenter is as follows:

$$Error ! Reference source not found . \frac{W \vert \frac{h}{R} = 0.1 or 0.5}{{Ch}^{3}} = {F}_{i} ( \frac{C}{E} , \frac{{C}_{r}}{C} ) . \qquad ( 6 )$$

where the shape function Πᵢ is affected by an elastic modulus E of a rock sample, a taper angle α of an indenter tip, a radius R of the indenter tip, a plasticity parameter fₚ of a rock material and microstructure characteristics fₚₒᵣₑ of a contact area between the indenter and rock sample.

Thereby, a functional relationship between loading and unloading curves of the micro indentation experiment and micro elastic-plastic parameters of the rock sample is established.

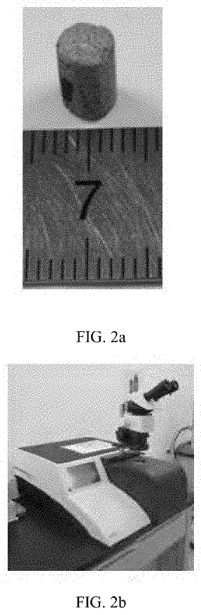

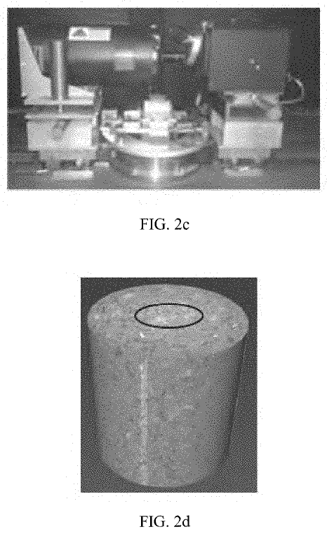

(2) A rock sample S1 which is a column with a diameter of 5 mm is prepared as shown in FIG. 2a, where upper and lower surfaces of the rock sample S1 are polished to horizontal and smooth by an Argon plasma as shown in FIG. 2b; the rock sample S1 is dried in a drying oven for 12 h at 65° C.; and a three-dimensional diagram of the rock sample S1, as shown in FIG. 2d, is obtained via a micro-CT scanner as shown in FIG. 2c. A reconstructed model including a micro rock matrix of the rock sample S1 with a size of 750×750×375 μm³ and a truncated cone-shaped indenter having a flat tip with a taper angle of 60° and a radius of 100 μm adopted in the experiment is shown in FIG. 3.

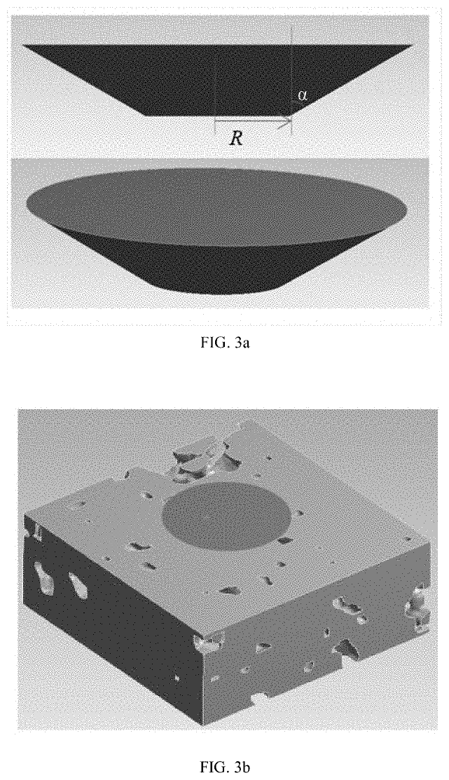

(3) The micro indentation experiment is carried out on the rock sample S1. As shown in FIG. 4, loading and unloading curves of the indenter are obtained by using a loading method of displacement control, where the maximum loading displacement is 15 μm. Micro elastic modulus of the rock sample is 17.8 GPa, which is calculated according to an indentation experiment specification. The work of the indenter is calculated at feature depths h/R respectively of 0.1 and 0.15.

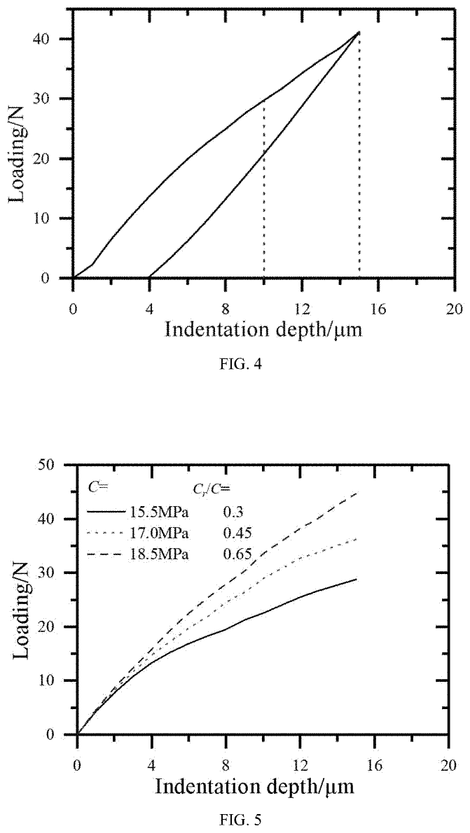

(4) The reconstructed model is imported into Mimics for meshing, and a micro indentation numerical simulation for the rock is performed by Ansys. In the numerical simulation, surfaces of the rock sample and the indenter have a friction coefficient of 0.15, following Coulomb law. A Poisson's ratio of the rock sample is obtained according to rock mechanic test results of a parallel sample. Research has shown that the friction coefficient and Poisson's ratio have little effect on loading and unloading curves of the micro indentation numerical simulation. A constitutive model of the rock sample meets the Mohr-Coulomb criterion, and the internal friction angles of the rock before and after failure are 46°, which are determined by a conventional indoor triaxial test. The initial cohesive force of the rock has a range of [14, 18.5] MPa, a ratio of the residual cohesive force to the initial cohesive force of the rock has a range of [0.3, 0.65]. The loading curves of the rock sample S1 under different strength characteristics in the numerical simulation are obtained as shown in FIG. 5.

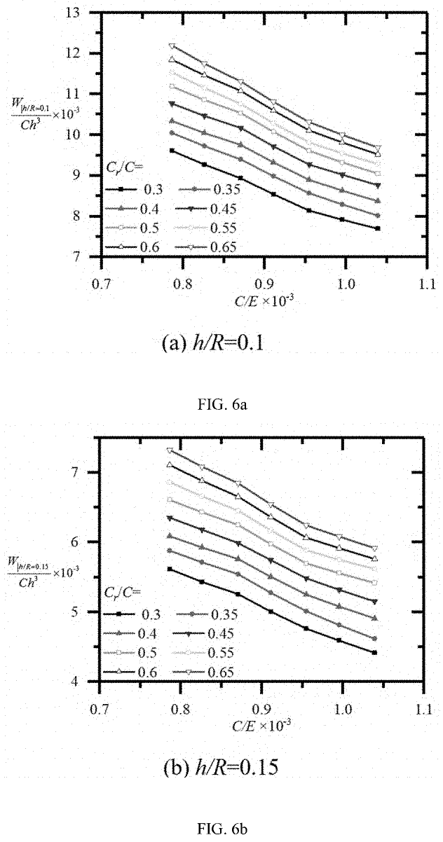

(5) A simulated work of the indenter obtained in the numerical simulation at the feature depths h/R respectively of 0.1 and 0.15 is calculated. Cᵣ/E and W/Ch³ are respectively used as horizontal coordinate and vertical coordinate to plot relation curves between W/Ch³ and Cᵣ/E as shown in FIG. 6. The simulated work of the indenter under different strengths and residual strengths is fitted via a cubic polynomial as follows:

$$\frac{W}{{Ch}^{3}} = {{A}_{4} [ ln ( \frac{C}{E} ) ]}^{3} + {{A}_{3} [ ln ( \frac{C}{E} ) ]}^{2} + {A}_{2} [ ln ( \frac{C}{E} ) ] + {A}_{1} \text{;} \qquad ( 7 )$$

where coefficients A₁˜A₄ are fitted according to simulation data, as shown in Table 1 to establish a relation function between W/Ch³ and Cᵣ/E under different strengths and residual strengths.

TABLE 1
Coefficients A₁~A₄ of the rock sample S1

| | | | | | |
|---|---|---|---|---|---|
| Cᵣ/C | A₁ | A₂ | A₃ | A₄ | R² |

(a) h/R = 0.1

| | | | | | |
|---|---|---|---|---|---|
| 0.3 | 22.16209 | 9.47943 | 1.35092 | 0.06412 | 0.99909 |
| 0.35 | 21.51197 | 9.22948 | 1.31927 | 0.0628 | 0.9991 |
| 0.4 | 28.44073 | 12.20165 | 1.74425 | 0.08306 | 0.99842 |
| 0.45 | 32.3675 | 13.87848 | 1.98291 | 0.09438 | 0.99812 |
| 0.5 | 30.59419 | 13.12187 | 1.87527 | 0.08927 | 0.99847 |
| 0.55 | 28.86454 | 12.35408 | 1.76177 | 0.08368 | 0.99888 |
| 0.6 | 29.27845 | 12.54496 | 1.79095 | 0.08516 | 0.99885 |
| 0.65 | 24.40938 | 10.44605 | 1.48933 | 0.07071 | 0.99932 |

(b) h/R = 0.15

| | | | | | |
|---|---|---|---|---|---|
| 0.3 | 12.59101 | 5.4104 | 0.77454 | 0.03693 | 0.99902 |
| 0.35 | 14.1728 | 6.10228 | 0.87534 | 0.04182 | 0.99873 |
| 0.4 | 15.47887 | 6.65299 | 0.95277 | 0.04545 | 0.99851 |
| 0.45 | 18.11635 | 7.78475 | 1.11466 | 0.05317 | 0.99795 |
| 0.5 | 22.04308 | 9.44551 | 1.34872 | 0.06416 | 0.99767 |
| 0.55 | 22.57485 | 9.66156 | 1.37788 | 0.06546 | 0.99787 |
| 0.6 | 20.32178 | 8.69793 | 1.24048 | 0.05893 | 0.99847 |
| 0.65 | 19.92019 | 8.52655 | 1.21608 | 0.05777 | 0.99864 |

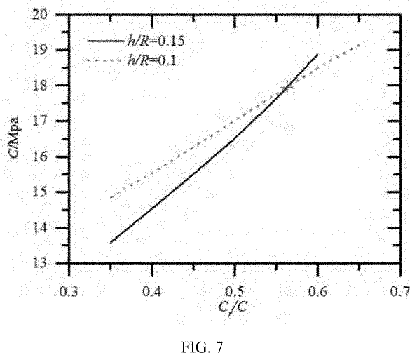

(6) The work of the indenter at the h/R of 0.1 and 0.15 obtained in the micro indentation experiment is substituted into Equation (7). As shown in FIG. 7, and the initial cohesive force C and the residual cohesive force Cᵣ are respectively used as vertical coordinate and horizontal coordinate to plot two curves at the h/R of 0.1 and 0.15; where the abscissa and ordinate of an intersection point of the two curves respectively represents a micro-scale initial cohesive force and the a micro-scale residual cohesive force of the rock sample in microscale. The micro-scale strength and the residual strength of the rock sample are obtained by the Mohr-Coulomb criterion.

Described above are merely preferred embodiments of the invention, which are intended to describe the technical solutions, characteristics and beneficial effects of the invention, and are not intended to limit the invention. Any modifications, replacements and variations made without departing from the spirit of the invention should fall within the scope of the invention.

What is claimed is:

1. A method for measuring micro-scale strength and residual strength of brittle rocks, comprising:
(1) performing a dimensionless analysis of a work of the indenter during a loading process before a micro indentation experiment;
(2) selecting and preparing a rock sample, obtaining the microstructure characteristics via CT scanning and reconstructing a finite element grid model of the rock matrix and the indenter in combination with a digital rock modeling technique;
(3) carrying out the micro indentation experiment on the rock sample to obtain micro elastic modulus of the rock sample according to an indentation experiment specification; and calculating the work of the indenter at different feature depths h/R;
(4) performing a micro indentation numerical simulation for the rock sample under different strengths and residual strengths based on the micro elastic modulus obtained in the micro indentation experiment to obtain loading and unloading curves of the numerical simulation;
(5) calculating a simulated work of the indenter obtained in the numerical simulation at the feature depths h/R respectively of 0.1 and 0.15; and fitting the simulated work of the indenter obtained under different strengths and residual strengths via a cubic polynomial to obtain a fitting formula;
(6) substituting values of the work of the indenter obtained at h/R respectively of 0.1 and 0.15 into the fitting formula; plotting two curves using an initial cohesive force C as vertical coordinate and a residual cohesive force Cᵣ as horizontal coordinate at the h/R respectively of 0.1 and 0.15, wherein an abscissa and ordinate of an intersection point of the two curves respectively represent a micro-scale initial cohesive force and a micro-scale residual cohesive force of the rock sample at a detection point; and obtaining the micro-scale strength and residual strength of the rock sample by the Mohr-Coulomb criterion.

2. The method of claim 1, wherein a shape function Πᵢ of a loading curve of the micro indentation experiment is expressed as function (1):
Πᵢ=Fᵢ(E,fₚ,α,h,R,fₚₒᵣₑ) (1);
wherein E is the elastic modulus of the rock sample, α is a taper angle of an indenter tip, R is a radius of the indenter tip, fₚ is a plasticity parameter of a rock material, h is an indentation depth and fₚₒᵣₑ is the microstructure characteristics of a contact area between the indenter and rock sample.

3. The method of claim 1, wherein the work of the indenter is expressed as equation (2):
W=∫₀^{h}^{ₘₐₓ}Fdh (2).

4. The method of claim 1, wherein a rock matrix of the brittle rocks is homogeneous and isotropic and meets the Mohr-Coulomb criterion (3):
τₙ=C+σₙ tan φ (3);
wherein τₙ is shear stress, σₙ is normal stress, C and φ are the initial cohesive force and an initial internal friction angle of the rock sample, respectively;
Cᵣ and φᵣ are the residual cohesive force and a residual internal friction angle of the rock sample after failure, respectively; when the rock sample is not smashed, the micro-scale internal friction angle equals to a core-scale value and C=Cᵣ, so equation (2) is rewritten as equation (4):
W=Fᵢ(E,C,Cᵣ,α,h,R,fₚₒᵣₑ) (4);

5. The method of claim 1, wherein the microstructure of the contact area between the indenter and rock sample is reconstructed using micro-CT scanning, and for an indentation experiment using a specially-shaped indenter, the dimensionless analysis of equation (4) is simplified according to the Buckinham's π-theorem as follows:
$$\frac{W}{{Ch}^{3}} = {F}_{i} ( \frac{C}{E} , \frac{{C}_{r}}{C} , \frac{h}{R} ) . \qquad ( 5 )$$

6. The method of claim 5, wherein when the feature depth h/R of the indentation experiment is set to 0.1 and 0.15, equation (5) is rewritten as function (6):
$$\frac{W \vert \frac{h}{R} = 0.1 or 0.5}{{Ch}^{3}} = {F}_{i} ( \frac{C}{E} , \frac{{C}_{r}}{C} ) . \qquad ( 6 )$$

7. The method of claim 5, wherein after the indentation experiment, the microstructure characteristics is obtained via a micro CT imaging technique and the finite element grid model of the rock matrix and the indenter is reconstructed in combination with a digital rock modeling technique.

8. The method of claim 1, wherein in step (3), the micro indentation experiment for the rock sample is carried out according to the indentation experiment specification to obtain the loading and unloading curves to calculate the micro elastic modulus of the rock sample.

9. The method of claim 6, wherein the work of the indenter at the feature depths h/R of 0.1 and 0.15 is calculated in combination with the micro elastic modulus of the rock sample.

10. The method of claim 1, wherein in step (4), the micro indentation numerical simulation under different strengths and residual strengths is performed by taking the micro elastic modulus obtained in the micro indentation experiment as an input parameter to obtain the loading and unloading curves of the numerical simulation.

11. The method of claim 10, wherein the simulated work of the indenter at the feature depths h/R of 0.1 and 0.15 is calculated according to the loading and unloading curves of the numerical simulation.

12. The method of claim 10, wherein the cubic polynomial for fitting the simulated work of the indenter under different strengths and residual strengths is:
$$\frac{W}{{Ch}^{3}} = {{A}_{4} [ ln ( \frac{C}{E} ) ]}^{3} + {{A}_{3} [ ln ( \frac{C}{E} ) ]}^{2} + {A}_{2} [ ln ( \frac{C}{E} ) ] + {A}_{1} \text{;} \qquad ( 7 )$$
wherein coefficients A₁˜A₄ are fitted according to simulation data.

13. The method of claim 1, wherein in step (6), the work of the indenter obtained in the micro indentation experiment at the h/R of 0.1 and 0.15 is substituted into equation (7), and the initial cohesive force C and the residual cohesive force Cᵣ are respectively used as vertical coordinate and horizontal coordinate to plot two curves at the h/R of 0.1 and 0.15; wherein the abscissa and ordinate of an intersection point of the two curves respectively represents the micro-scale initial cohesive force and the micro-scale residual cohesive force of the rock sample.

14. The method of claim 13, wherein the micro-scale strength and the residual strength of the rock sample are obtained by substituting the micro-scale initial cohesive force and the micro-scale residual cohesive force of the rock sample into equation (3).
